(19) United States
(12) Patent Application Publication
(10) Pub. No.: US 2026/0198997 A1
(43) Pub. Date: Jul. 16, 2026

(54) Tissue Removal Systems and Related Methods

(71) Applicant: Madison Surgical Designs, LLC, La Canada, CA (US)

(72) Inventor: Behnam Badie, La Canada, CA (US)

(73) Assignee: Madison Surgical Designs, LLC, La Canada, CA (US)

(21) Appl. No.: 19/544,614

(22) Filed: Feb. 19, 2026

Related U.S. Application Data
(63) Continuation-in-part of application No. PCT/US2025/042189, filed on Aug. 15, 2025.
(60) Provisional application No. 63/683,432, filed on Aug. 15, 2024.

(51) Int. Cl. A61B 18/14; A61B 18/00

(52) U.S. Cl. CPC A61B 18/1482; A61B 2018/00202; A61B 2018/00577; A61B 2018/00601; A61B 2018/00994; A61B 2018/141; A61B 2218/002; A61B 2218/007

(57) ABSTRACT

Tissue removal systems and related methods employ remote actuation of a tissue removal device. A tissue removal system can include a tissue removal device, a tissue cutting wire drive cable assembly, a tissue cutting tube drive cable assembly, and a control unit. The control unit can include a tissue cutting wire drive motor and a tissue cutting tube drive motor. The tissue cutting wire drive motor can remotely drive actuation of tissue cutting wires of the tissue removal device via actuation of the tissue cutting wire drive cable assembly. The tissue cutting tube drive motor can remotely drive actuation of a tissue cutting tube of the tissue removal device via actuation of the tissue cutting tube drive cable assembly.

### CROSS-REFERENCES TO RELATED APPLICATIONS

The present application is a Continuation-in-Part of PCT Patent Appln. No. PCT/US2025/042189 filed Aug. 15, 2025; which claims the benefit of U.S. Provisional Appln. No. 63/683,432 filed Aug. 15, 2024, the full disclosures which are incorporated herein by reference in their entirety for all purposes.

### BACKGROUND

Brain tumors account for 85% to 90% of all primary central nervous system (CNS) tumors (see, e.g., Levin V.A., et al., Cancer: Principles and Practice of Oncology. 6th ed. Philadelphia, Pa: Lippincott Williams & Wilkins, 2001, pp 2100-60; herein incorporated by reference in its entirety). Available registry data from the Surveillance, Epidemiology, and End Results (SEER) database for 1996 to 2000 indicate that the combined incidence of primary invasive CNS tumors in the United States is 6.6 per 100,000 persons per year, with an estimated mortality of 4.7 per 100,000 persons per year (see, e.g., Trends in SEER incidence and U.S. mortality using the joinpoint regression program 1975-2000 with up to three joinpoints by race and sex. In: Ries LAG, Eisner MP, Kosary CL, et al.: SEER Cancer Statistics Review, 1975-2000. Bethesda, Md: National Cancer Institute, 2003; herein incorporated by reference in its entirety). Worldwide, approximately 176,000 new cases of brain and other CNS tumors were diagnosed in the year 2000, with an estimated mortality of 128,000 (see, e.g., Parkin D.M., et al., Int J Cancer 94 (2): 153-6, 2001; herein incorporated by reference in its entirety).

Metastatic tumors are among the most common mass lesions in the brain. In recent years, the incidence of CNS metastasis has increased. This is because, for example, the median survival duration of cancer patients has increased as a result of modem therapies, increased availability of advance imaging techniques, and vigilant surveillance protocols. Unfortunately, some chemotherapeutic agents can weaken the blood-brain barrier (BBB) transiently and allow CNS seeding. Moreover, a number of commonly used chemotherapeutic agents do not cross the BBB, thus leaving the brain as a safe haven for tumor growth. Metastases from systemic cancer can affect brain parenchyma, its covering, and the skull. Different tumors metastasize to different organs preferentially. Generally, cells with similar origins are believed to have similar growth constraints and to embryologically express similar sets of adhesive molecules such as addressins. In the United States, incidence of metastatic brain tumor is exceeding that of primary brain tumor. Metastatic brain tumors comprise 50% of all brain tumors and as many as 30% of tumors diagnosed by imaging study alone. The incidence is estimated to be 100,000 new cases per year in the United States. In autopsy studies, over 20% of patients with systemic neoplastic disease have brain metastasis.

The clinical presentation of various brain tumors is best appreciated by considering the relation of signs and symptoms to anatomy (see, e.g., Levin V.A., et al., Cancer: Principles and Practice of Oncology. 6th ed. Philadelphia, Pa: Lippincott Williams & Wilkins, 2001, pp 2100-60; herein incorporated by reference in its entirety). General signs and symptoms include headache, gastrointestinal symptoms (e.g., nausea, loss of appetite, and vomiting) and changes in personality (e.g., changes in mood, mental capacity, and concentration). Whether primary, metastatic, malignant, or benign, brain tumors must be differentiated from other space-occupying lesions such as abscesses, arteriovenous malformations, and infarction, which can have a similar clinical presentation (see, e.g., Hutter A, et al., Neuroimaging Clin N Am 13 (2): 237-50, x-xi, 2003; herein incorporated by reference in its entirety).

Surgery is the treatment of choice for accessible brain tumors. Accessible tumors are those that can be surgically removed without causing severe neurological damage.

Deeply seated tumors (e.g., brain tumors located in the brain stem, the thalamus, the motor area, and the deep areas of gray matter) may be inaccessible, and as such, inoperable. The goal of surgery is to remove all or most of the visible tumor. Many benign tumors are treated only by surgery. Most malignant tumors require additional treatment. Malignant tumors lack distinct borders. They often invade nearby normal brain tissue. Tumor cells may also spread throughout the brain and spine by way of the cerebrospinal fluid. But even partial tumor removal is beneficial.

There are several purposes of brain tumor related neurosurgery. One purpose of brain tumor related surgery is to remove as much tumor as possible. Partial brain tumor removal (e.g., debulking) provides relief of symptoms, improved quality of life, and a smaller tumor burden for other treatment modalities. Brain tumor related neurosurgery also assists in establishing an exact diagnosis. For example, removal of a sample of tumor (e.g., a tumor biopsy) to be examined under a microscope in the laboratory provides an exact diagnosis. Furthermore, brain tumor related neurosurgery provides access for other treatments. For example, during neurosurgery radiation implants or chemotherapy-impregnated wafers may be delivered to the brain tumor. Biopsy alone is performed when the tumor is inoperable or when surgery must be delayed. Resection (e.g., surgical removal of a tumor) is the treatment of choice whenever possible.

Neurosurgery, however, demands special considerations. Obtaining surgical access to brain tumors requires the creation of an opening in the skull (called a craniotomy). Most often, a craniotomy involves a large incision and dissection of other soft tissue that results in significant postoperative pain and discomfort. Furthermore, reaching deep tumors in the brain requires openings into the surface of the brain itself. This brain dissection and manipulation can result in neurological deficits.

What is needed are improved neurosurgical techniques for accessing brain locations. Additionally, what are needed are improved devices assisting in neurosurgical techniques that limit soft tissue dissection and potential brain manipulation and damage.

Additionally, what is needed are improved devices for cutting, cauterizing and aspirating brain tumors through small openings in the skull and brain tissue.

### BRIEF SUMMARY

The following presents a simplified summary of some tissue removal systems and tissue removal methods of the present disclosure in order to provide a basic understanding of the tissue removal systems and tissue removal methods. This summary is not an extensive overview of the tissue removal systems and tissue removal methods of the present disclosure. It is not intended to identify key/critical elements of the tissue removal systems and tissue removal methods of the present disclosure or to delineate the scope of the tissue removal systems and tissue removal methods of the present disclosure. Its sole purpose is to present some tissue removal systems and tissue removal methods of the present disclosure in a simplified form as a prelude to the more detailed description that is presented later.

Examples described herein are directed to tissue removal systems and related methods. A tissue removal system can include a tissue removal device that includes one or more expandable tissue cutting wires that are remotely actuated via rotary actuation cables that supply rotational inputs to the tissue removal device. The remote actuation of the one or more tissue cutting wires enables the tissue removal device to be configured without actuation motors, thereby enhancing the ability to sterilize the tissue removal device between procedures and/or to enable the usage of magnetic resonance imaging of the tissue during with the tissue removal device. The tissue removal device can be configured for insertion of an ultrasound transducer into the tissue removal device for imaging of the one or more tissue cutting wires and targeted tissue during cutting of the targeted tissue via rotation and expansion of the one or more tissue cutting wires. The ultrasound transducer can be removed to accommodate irrigation and aspiration, thereby enhancing removal of the tissue cut by the one or more tissue cutting wires.

Thus, in one aspect, a tissue removal system includes a tissue removal device, a tissue cutting wire drive cable assembly, a tissue cutting tube drive cable assembly, and a control unit. The tissue removal device includes a tissue cutting wire support tube, a tissue cutting tube, one or more tissue cutting wires, a tissue cutting wire actuation mechanism, and a tissue cutting tube actuation mechanism. The tissue cutting wire support tube has a tissue cutting wire support tube lumen. The tissue cutting tube has a tissue cutting tube lumen. The tissue cutting tube is disposed within the tissue cutting wire support tube lumen. The one or more tissue cutting wires are configured to be rotated to cut a tissue. The tissue cutting wire actuation mechanism includes a tissue cutting wire drive input. The tissue cutting wire actuation mechanism is configured to rotate and outwardly expand the one or more tissue cutting wires in response to rotation of the tissue cutting wire drive input in a first direction. The tissue cutting wire actuation mechanism is configured to rotate and inwardly contract the one or more tissue cutting wires in response to rotation of the tissue cutting wire drive input in a second direction opposite to the first direction. The tissue cutting tube actuation mechanism includes a tissue cutting tube drive input. The tissue cutting tube actuation mechanism is configured to rotate the tissue cutting tube in response to rotation of the tissue cutting tube drive input. The tissue cutting wire support tube has tissue cutting wire support tube tissue removal openings. The tissue cutting tube has tissue cutting tube radial openings configured to cooperate with the tissue cutting wire support tube tissue removal openings to chop tissue and allow aspiration of the chopped tissue through the tissue cutting tube lumen. The tissue cutting wire drive cable assembly is drivingly coupled with the tissue cutting wire drive input. The tissue cutting tube drive cable assembly is drivingly coupled with the tissue cutting tube drive input. The control unit includes a tissue cutting wire drive output, a tissue cutting wire drive motor drivingly coupled with the tissue cutting wire drive output, a tissue cutting tube drive output, and a tissue cutting tube drive motor drivingly coupled with the tissue cutting tube drive output. The tissue cutting wire drive output is configured to be drivingly coupled with the tissue cutting wire drive cable assembly. The tissue cutting tube drive output is configured to be drivingly coupled with the tissue cutting tube drive cable assembly. The control unit is configured to control operation of the tissue cutting wire drive motor to control rotation, expansion, and contraction of the one or more tissue cutting wires. The control unit is configured to control operation of the tissue cutting tube drive motor to control rotation of the tissue cutting tube.

The tissue removal system can be configured for use with magnetic resonance imaging. For example, one or more of the tissue removal device, the tissue cutting wire drive cable assembly, or the tissue cutting tube drive cable assembly can be made of one or more magnetic resonance imaging compatible materials to accommodate usage of magnetic resonance imaging with the tissue removal device.

The tissue removal system can be configured for use with ultrasound imaging. For example, the tissue removal system can further include an ultrasound transducer for imaging the one or more tissue cutting wires during cutting of the tissue via rotation and expansion of the one or more tissue cutting wires. The ultrasound transducer can be insertable into the tissue cutting tube lumen to a position for the imaging of the one or more tissue cutting wires. Each of the tissue removal device, the tissue cutting wire drive cable assembly, the tissue cutting tube drive cable assembly, and the ultrasound transducer can be made of one or more magnetic resonance imaging compatible materials to accommodate usage of magnetic resonance imaging with the tissue removal device.

The tissue removal system can be configured to aspirate tissue. For example, the tissue removal device can further include an aspiration port. The tissue removal system can further include a tissue aspiration assembly in fluid communication with the aspiration port and operable to aspirate the tissue cut by the one or more tissue cutting wires. The tissue aspiration assembly can further include a tissue aspiration tube configured to be fluidly coupled with the aspiration port for aspiration of the tissue cut by the one or more tissue cutting wires.

The tissue removal system can be configured to irrigate tissue. For example, the tissue removal system can further include an irrigation assembly configured to supply an irrigation fluid. The tissue removal device can further include an irrigation port configured to receive the irrigation fluid. The tissue removal device can be configured to output the irrigation fluid to irrigate the tissue cut by the one or more tissue cutting wires and/or a tissue bordering the tissue cut by the one or more tissue cutting wires. The irrigation assembly can further include an irrigation fluid source.

The tissue removal system can be configured to simultaneously irrigate and aspirate tissue. For example, the tissue removal system can further include a tissue aspiration assembly and an irrigation assembly. The tissue aspiration assembly can be operable to aspirate the tissue cut by the one or more tissue cutting wires. The irrigation assembly can be configured to supply an irrigation fluid. The tissue removal device can further include an irrigation port configured to receive the irrigation fluid. The tissue removal device can be configured to output the irrigation fluid to irrigate the tissue cut by the one or more tissue cutting wires and/or a tissue bordering the tissue cut by the one or more tissue cutting wires.

In the tissue removal system, relative rotation between the tissue cutting tube and the tissue cutting wire support tube can be used to chop aspirated tissue. For example, the tissue cutting wire support tube can have tissue cutting wire support tube tissue removal openings. The tissue cutting tube can have tissue cutting tube radial openings configured to cooperate with the tissue cutting wire support tube tissue removal openings to chop tissue and allow aspiration of the chopped tissue through the tissue cutting tube lumen.

In the tissue removal system, the tissue cutting wire actuation mechanism can be operable to expand and contract the one or more tissue cutting wires. For example, the tissue cutting wire actuation mechanism can be operable to expand the one or more tissue cutting wires by moving proximal ends of the one or more tissue cutting wires toward a distal end of the tissue cutting wire support tube. The tissue cutting wire actuation mechanism can be operable to contract the one or more tissue cutting wires by moving the proximal ends of the one or more tissue cutting wires away from the distal end of the tissue cutting wire support tube.

The tissue removal system can be configured to apply radiofrequency (RF) energy to the tissue via at least one of the one or more tissue cutting wires. For example, the tissue removal system can further include an energy generator connection operatively coupled with at least one of the one or more tissue cutting wires for transferring RF energy from an RF energy source to the at least one of the one or more tissue cutting wires.

The tissue removal system can include a cannula that includes one or more ultrasound transducers that are used to image the tissue during removal of the tissue via operation of the tissue removal device. For example, the tissue removal system can include a cannula having a distal end and configured to be held in a fixed position relative to the tissue. A distal end of the tissue removal device can be inserted through the cannula to position the tissue removal device for removal of the tissue. The cannula can include one or more ultrasound transducers of an ultrasound imaging system operable to image the tissue during removal of the tissue via operation of the tissue removal device. As another example, the tissue removal system can include a cannula and a mounting fixture. The cannula can have a distal end and can be configured to be held in a fixed position relative to the tissue to be removed. A distal end of the tissue removal device can be insertable through the cannula to position the tissue removal device for removal of the tissue. The cannula can include one or more cannular ultrasound transducers of an ultrasound imaging system operable to image the tissue during removal of the tissue via operation of the tissue removal device. The mounting fixture can have any suitable configuration for supporting the tissue removal device in a suitable position and orientation relative to the tissue. For example, the mounting fixture can be configured for mounting to a patient having the tissue to be removed. As another example, the mounting fixture can be part of a robotic assembly or a support assembly configured for supporting the tissue removal device in a suitable position and orientation relative to the tissue to be removed. The mounting fixture can include a lumen through which the cannula is insertable to position the distal end of the cannula relative to the tissue to be removed. The mounting fixture can include one or more mounting fixture ultrasound transducers of the ultrasound imaging system.

The tissue removal system can include an auxiliary ablation device that can be used in conjunction with the tissue removal device to remove the tissue. For example, the tissue removal system can include a cannula and an auxiliary ablation device. The cannula can have a distal end and can be configured to be held in a fixed position relative to the tissue to be removed. A distal end of the tissue removal device can be inserted through the cannula to position the tissue removal device for removal of the tissue. The auxiliary ablation device can include an auxiliary ablation assembly operable to ablate the tissue. The auxiliary ablation assembly can be inserted through the cannula to position the auxiliary ablation assembly adjacent the tissue for ablation of the tissue by the auxiliary ablation assembly.

The auxiliary ablation device can employ any suitable ablation technology. For example, the auxiliary ablation assembly can include: (a) a Laser Interstitial Thermal Therapy (LITT) ablation assembly operable to ablate the tissue via LITT, (b) a Magnetic Resonance Imaging Guided Focused Ultrasound (MRgFUS) ablation assembly operable to ablate the tissue via MRgFUS, (c) a Radiofrequency Ablation (RFA) assembly operable to ablate the tissue via RFA, (d) a Microwave Ablation (MWA) assembly operable to ablate the tissue via MWA, (e) a Cryoablation Ablation assembly operable to ablate the tissue via cyroablation, and/or (f) an Irreversible Electroporation (IRE) assembly operable to ablate the tissue via IRE.

In another aspect, a method of removing a targeted tissue from a patient is provided. The method includes: (a) positioning a distal end of a cannula relative to the targeted tissue; (b) inserting a distal end of a tissue removal device through the cannula to position one or more tissue cutting wires of the tissue removal device adjacent to or within the targeted tissue; (c) controlling, via a control unit, a tissue cutting wire drive motor to rotate a rotatable member of a tissue cutting wire drive cable assembly to rotate a tissue cutting wire drive input of the tissue removal device to simultaneously rotate and expand the one or more tissue cutting wires to cut the targeted tissue; (d) controlling, via the control unit, the tissue cutting wire drive motor to rotate the rotatable member of the tissue cutting wire drive cable assembly to rotate the tissue cutting wire drive input of the tissue removal device to simultaneously rotate and contract the one or more tissue cutting wires; (e) outputting irrigation fluid from the tissue removal device to irrigate the targeted tissue; and (f) aspirating at least some of the targeted tissue via the tissue removal device.

In the method of removing a targeted tissue, the tissue removal device can chop the targeted tissue. For example, the tissue removal device can include a tissue cutting wire support tube and a tissue cutting tube disposed within the tissue cutting wire support tube. The tissue cutting wire support tube can have tissue cutting wire support tube tissue removal openings. The tissue cutting tube can have a tissue cutting tube lumen and tissue cutting tube radial openings configured to cooperate with the tissue cutting wire support tube tissue removal openings to chop tissue and accommodate aspiration of the chopped tissue through the tissue cutting tube lumen. The method can include controlling, via the control unit, a tissue cutting drive motor to rotate a rotatable member of a tissue cutting tube drive cable assembly to rotate a tissue cutting drive input of the tissue removal device to rotate the tissue cutting tube relative to the tissue cutting wire support tube.

The method of removing a targeted tissue can include magnetic resonance imaging. For example, the method of removing a target tissue can further include conducting magnetic resonance imaging of the targeted tissue during operation of the tissue removal device. Each of the tissue removal device, the tissue cutting wire drive cable assembly, and the tissue cutting tube drive cable assembly can be made of one or more magnetic resonance imaging compatible materials.

The method of removing a targeted tissue can include ultrasound imaging of the targeted tissue. For example, the method can include conducting ultrasound imaging of the one or more tissue cutting wires via an ultrasound transducer disposed within the tissue removal device during cutting of the targeted tissue via rotation and expansion of the one or more tissue cutting wires. The method can further include inserting the ultrasound transducer into the tissue removal device prior to the cutting of the targeted tissue via rotation and expansion of the one or more tissue cutting wires. The method can further include removing the ultrasound transducer from the tissue removal device prior to rotation and contraction of the one or more tissue cutting wires and coupling an aspiration assembly to the tissue removal device for aspiration of the targeted tissue.

The method of removing a targeted tissue can include applying radio frequency (RF) energy to the targeted tissue. For example, the method can further include operating an RF energy source to supply RF energy to at least one of the tissue cutting wires to apply RF energy to the targeted tissue.

The method of removing a targeted tissue can include imaging the tissue during removal of the tissue via operation of the tissue removal device. For example, the imaging of the tissue can be accomplished via an ultrasonic imaging system comprising one or more ultrasound transducers mounted to the cannula. As another example, the imaging of the tissue can be accomplished via an ultrasonic imaging system comprising one or more cannula ultrasound transducers mounted to the cannula and one or more mounting fixture ultrasound transducers mounted to a mounting fixture configured to support the tissue removal device in a suitable position and orientation relative to the tissue to be removed. For example, the mounting fixture can be configured to be attached to a patient having the tissue to be removed. As another example, the mounting fixture can be part of a robotic assembly or support assembly configured for supporting the tissue removal device in a suitable position and orientation relative to the tissue to be removed. The mounting fixture can include a lumen through which the cannula extends at least partially.

The method of removing a targeted tissue can include operating an auxiliary ablation device to ablate the tissue. The auxiliary ablation device can include an auxiliary ablation assembly that is positioned relative to the tissue through the cannula.

The auxiliary ablation device operated in the method can employ any suitable ablation technology. For example, the auxiliary ablation assembly can include: (a) a Laser Interstitial Thermal Therapy (LITT) ablation assembly operable to ablate the tissue via LITT, (b) a Magnetic Resonance Imaging Guided Focused Ultrasound (MRgFUS) ablation assembly operable to ablate the tissue via MRgFUS, (c) a Radiofrequency Ablation (RFA) assembly operable to ablate the tissue via RFA, (d) a Microwave Ablation (MWA) assembly operable to ablate the tissue via MWA, (e) a Cryoablation Ablation assembly operable to ablate the tissue via cyroablation, and/or (f) an Irreversible Electroporation (IRE) assembly operable to ablate the tissue via IRE.

For a fuller understanding of the nature and advantages of the tissue removal systems and tissue removal methods of the present disclosure, reference should be made to the ensuing detailed description and accompanying drawings.

### BRIEF DESCRIPTION OF THE DRAWINGS

FIG. 1 illustrates a sequence of steps that can be used to remove a targeted tissue using a tissue removal device and related components, in accordance with tissue removal systems and tissue removal methods of the present disclosure.

FIG. 2 schematically illustrates a first configuration of a tissue removal system that includes the tissue removal device of FIG. 1, in accordance with tissue removal systems and tissue removal methods of the present disclosure.

FIG. 3 schematically illustrates a second configuration of the tissue removal system of FIG. 2.

FIG. 4 shows the tissue removal device of FIG. 1.

FIG. 5 shows a distal end portion of the issue removal device of FIG. 1 in an expanded configuration of tissue cutting wires.

FIG. 6 illustrates actuation related aspects of the tissue removal device of FIG. 1.

FIG. 7 schematically illustrates tissue cutting wire actuation and cutting tube actuation related components of the tissue removal device of FIG. 1 in a contracted configuration of tissue cutting wires.

FIG. 8 schematically illustrates the tissue cutting wire actuation and cutting tube actuation related components of FIG. 7 in an expanded configuration of the tissue cutting wires.

FIG. 9 schematically illustrates the tissue cutting wire actuation and the cutting tube actuation related components of FIG. 7 with an ultrasound transducer positioned for imaging of the tissue cutting wires and/or the targeted tissue.

FIG. 10 shows a simplified schematic flow chart of a method of removing a targeted tissue from a patient, in accordance with tissue removal systems and tissue removal methods of the present disclosure.

FIG. 11 schematically illustrates ultrasound transducers integrated with a mounting fixture and a cannula, in accordance with the systems and methods of the present disclosure.

FIG. 12 schematically illustrates an auxiliary ablation system that can be employed in conjunction with the tissue removal systems of the present disclosure.

### DETAILED DESCRIPTION

In the description herein, various tissue removal systems and tissue removal methods of the present disclosure are described. For purposes of explanation, specific configurations and details are set forth in order to provide a thorough understanding of the tissue removal systems and tissue removal methods of the present disclosure. However, it will also be apparent to one skilled in the art that the tissue removal systems and tissue removal methods of the present disclosure may be practiced without the specific details. Furthermore, well-known features may be omitted or simplified in order not to obscure the embodiment being described.

Examples described herein relate to tissue removal systems and tissue removal methods. The tissue removal systems and tissue removal methods of the present disclosure described herein can be used with respect to any suitable tissue, such as for contacting and treating brain tissue (e.g., brain tumors). The tissue removal systems and related methods of use may be described in the context of neurosurgical applications (e.g., brain tumor resection, brain tumor biopsy, brain tumor imaging, brain hematoma evacuation, decompression of contused or damaged brain tissue. It should be appreciated, however, that the tissue removal systems and related methods are not limited to neurosurgical applications but can be employed in any suitable procedure (e.g., spinal surgery, bone marrow applications, liver tumor surgery, etc.).

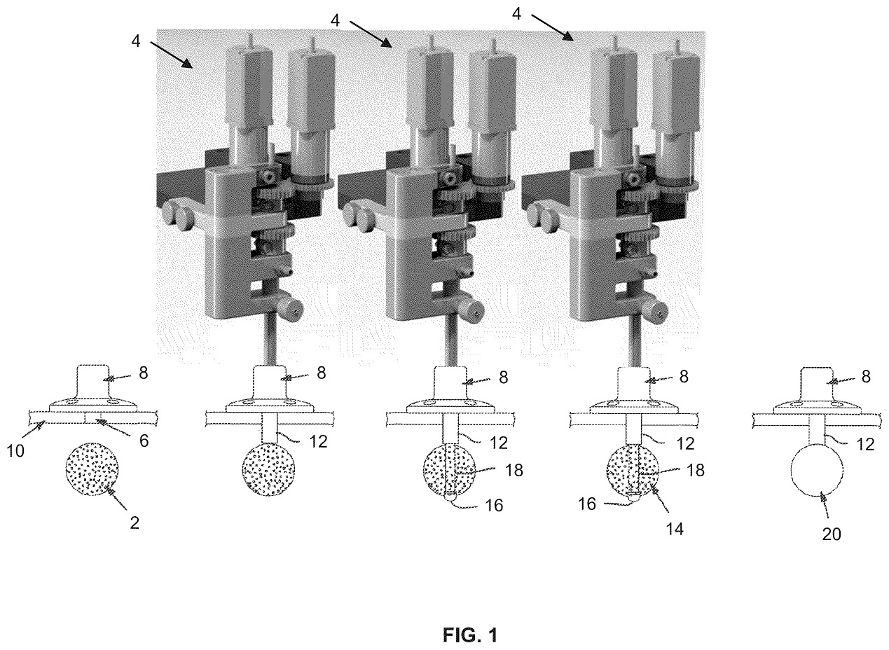

Turning now to the drawing figures in which similar reference numbers refer to similar features in the various drawing figures, FIG. 1 illustrates a sequence of steps that can be used to remove a brain mass 2 using a tissue removal device 4 and related components, in accordance with tissue removal systems and tissue removal methods of the present disclosure.

A standard burr hole 6 measuring 7-14 mm in diameter can be created. FIG. 1 shows an example approach in which a mounting fixture 8 is secured in place to a skull 10 for use in constraining the position and orientation of the tissue removal device 4 relative to the skull 10. Any suitable approach can be used for constraining the position and orientation of the tissue removal device 4 relative to the skull 10. For example, the mounting fixture can alternatively be part of a robotic assembly or a support assembly configured to support the tissue removal device 4 in a suitable position and orientation relative to the tissue to be removed. The approach employed for constraining the position and orientation of the tissue removal device 4 relative to the skull 10 can be based on specifics of the tissue removal device 4 and any medical imaging and/or navigation systems employed.

A cannula 12 configured to provide access to the brain mass 2 and allow for passage of the distal end portion of the tissue removal device 4 can be inserted through the cannula 12. The cannula 12 can have any suitable dimensions (e.g., inner diameter of 0.5 to 1 cm; length 2-5 cm). The cannula 12 can have a stylet that can be removed after inserting the cannula 12 into the burr hole 6. The cannula 12 can be made of advanced engineered polymers such as PEEK known for its high strength and biocompatibility. The cannula 12 can be inserted using image guidance techniques (such as Stealth or BrainLab) up to the edge of the brain mass 2.

The tissue removal device 4 (which includes expandable/contractable tissue cutting wires 14) cutting wires 674) can be introduced next through the cannula 12. The tissue removal device 4 is used for separation, fragmentation, and aspiration of the brain mass 2 (e.g., a tumor, a lesion, or a hematoma) from surrounding brain tissue. A tip 16 of the tissue removal device 4 can be inserted through the brain mass 2 to position the tip 16 adjacent to a distal surface of the brain mass 2. The tissue removal device 4 is then secured in place relative to the skull 10. The tissue removal device 4 includes a rotatable tissue cutting wire support tube 18 to which the tissue cutting wires 14 are coupled. As described herein, the tissue removal device 4 is operable to rotate and expand the tissue cutting wires 14 to an expanded configuration (e.g., circular or any other suitable expanded configuration) and to rotate and contract the tissue cutting wires 14 to a non-expanded configuration suitable for removal of the tissue removal device 4. The tissue cutting wires 14 can be metallic and connected to a radiofrequency (RF) source for application of RF energy to the brain mass 2. The tissue removal device 4 can be operated to extend the tissue cutting wires 14 within the brain mass 2, or beyond the brain mass 2, to accomplish disconnection of the brain mass 2 from surrounding hematoma or gliotic margin (in case of tumors) using RF energy.

The tissue removal device 4 can be removed and a balloon device that includes balloon 20 can be introduced. The balloon 20 is used for hemostasis and tamponade of small bleeding vessel. The balloon 20 is inserted through the cannula 12 and inflated to the appropriate volume to expand and completely fill the cavity. Pressure from the balloon 20 is sufficient to occlude small arterioles. Furthermore, the center of the balloon device can have a hollow catheter for injection of thrombogenic agents such as thrombin, which will flow over the surface of the balloon to promote hemostasis. The hollow catheter of the balloon device can be used for aspiration of the cavity contents, after the balloon 20 has been deflated. The hollow catheter of the balloon device can be connected to a pressure transducer to monitor intracranial pressure.

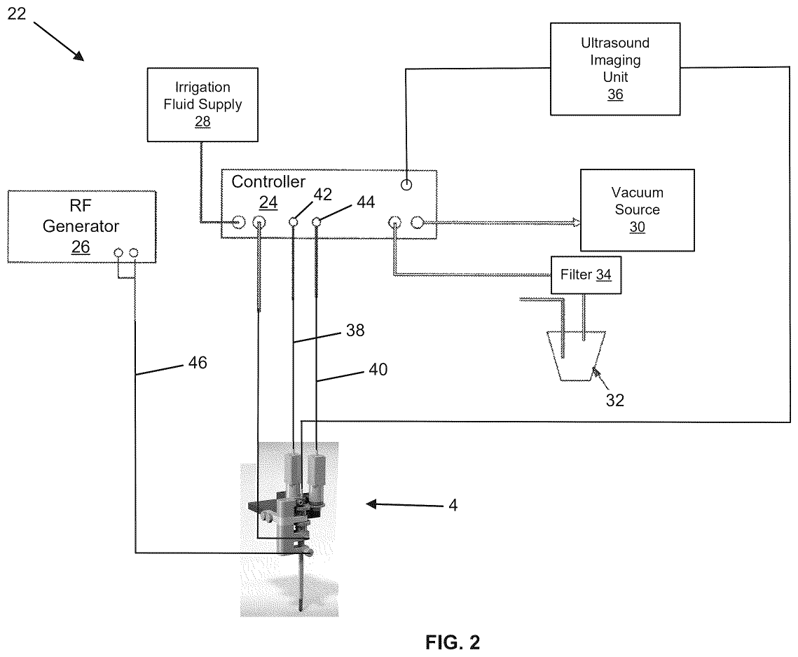

FIG. 2 schematically illustrates a first configuration of a tissue removal system 22, in accordance with tissue removal systems and tissue removal methods of the present disclosure. The tissue removal system 22 includes the tissue removal device 4, a controller 24, a radiofrequency (RF) generator 26, an irrigation fluid supply 28, a vacuum source 30, a tissue collection container 32, a filter 34, an ultrasound imaging unit 36, a tissue cutting wire drive cable assembly 38, and a tissue cutting tube drive cable assembly 40. The controller 24 inter-connects the tissue removal device 4, the RF generator 26, the irrigation fluid supply 28, and the vacuum source 30. The controller 24 contains electronic as well as electro-pneumatic interfaces and controlling mechanisms to facilitate cutting, cauterizing, fragmenting, and aspirating a targeted tissue. Different modes and sequences of operation are selected through front panel controls of the controller 24 by a surgeon. The tissue removal device 4 can include a pressure transducer operatively coupled with the controller 24 to measure and control the intracranial pressure as described herein.

The rotary drive cable assemblies 38, 40 are used to transmit rotational actuation inputs to the tissue removal device 4. The controller 24 includes a tissue cutting wire drive motor 42 and a tissue cutting tube drive motor 44. The tissue cutting wire drive motor 42 is drivingly coupled with the tissue cutting wire drive cable assembly 38. The tissue cutting tube drive motor 44 is drivingly coupled with the tissue cutting tube drive cable assembly 40. The tissue cutting wire drive motor 42 generates a tissue cutting wire actuation input that is supplied to the tissue removal device 4 by the tissue cutting wire drive cable assembly 38. The tissue cutting wire actuation input is used to produce rotation and expansion of the tissue cutting wires 14 and rotation and contraction of the tissue cutting wires 14 as described herein. The tissue cutting tube drive motor 44 generates a tissue cutting tube drive input that is supplied to the tissue removal device 4 by the tissue cutting tube drive cable assembly 40. The tissue cutting tube drive input is used to produce rotation of a tissue chopping tube of the tissue removal device 4 as described herein. The usage of the rotary drive cables 38,40 to actuate the actuated features of the tissue removal device 4 enables remove mounting of the drive motors 42, 44, thereby simplifying sterilization of the tissue removal device 4 and/or enabling the usage of magnetic resonance imaging in conjunction with the use of the tissue removal device 4 via construction of the tissue removal device 4 and the drive cable assemblies 38, 40 from suitable non-magnetic materials compatible with magnetic resonance imaging.

The tissue removal device 4 is electrically coupled with the RF generator 26 via an RF energy connection cable 46. The tissue removal device 4 is configured so that RF energy received via the connection cable 46 is transferred to the tissue cutting wires 14 for application to the targeted tissue. An exemplary RF generator is manufactured by PEAK Surgical (Palo Alto, CA). The PULSAR® generator supplies short pulsed electrical discharges and allows the tissue cutting wires 14 to cut at much lower average temperatures than conventional electrosurgery without sticking.

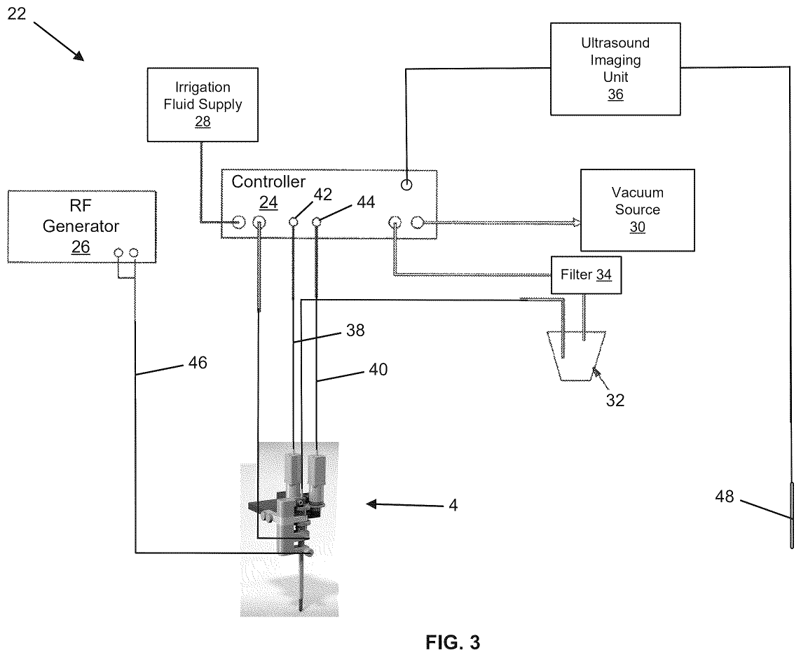

In the first configuration illustrated in FIG. 2, an ultrasound transducer 48 (shown in FIG. 3) operatively connected to the ultrasound unit 36 is disposed within the tissue removal device 4 at a position for ultrasound imaging of the targeted tissue and the tissue cutting wires 14. The first configuration may be employed during cutting of the targeted tissue via expansion of the tissue cutting wires 14 to monitor the cutting of the targeted tissue by the tissue cutting wires 14 for use in controlling operation of the tissue removal device 4. In the first configuration illustrated in FIG. 2, the tissue collection container 32 is not connected with the tissue removal device 4 to accommodate the positioning of the ultrasound transducer within the tissue removal device 4. Alternatively, the tissue removal device 4 can be configured to accommodate the ultrasound transducer concurrently with aspiration of the cut targeted tissue to the tissue collection container 32.

In the second configuration illustrated in FIG. 3, the tissue removal system 22 is configured and operable to conduct simultaneous irrigation and aspiration to remove the targeted tissue. The controller 24 is configured to draw irrigation fluid from the irrigation fluid supply 28 and transfer the irrigation fluid to the tissue removal device 4. The tissue removal device 4 outputs the irrigation fluid onto the targeted tissue, the cut targeted tissue, and the exposed tissue surrounding the cut targeted tissue. The vacuum source 30 generates a vacuum pressure that is supplied to the controller 24. The controller 24 controls the transmission of the vacuum pressure to the tissue collection container 32, which draws tissue and/or fluid from the tissue removal device 4 into the tissue collection container 32. The filter 34 filters air removed from the tissue collection container 32 to prevent contamination of the controller 24 and the vacuum source 30. The controller 24 can be configured to control the flow rate of the irrigation fluid to the tissue removal device 4 and the flow rate of tissue/fluid aspirated via the tissue removal device 4 to maintain a suitable pressure level within the patient at the targeted tissue site. The tissue removal device 4 can include a pressure sensor that supplies a pressure signal to the controller 24 for use by the controller to control the supply of irrigation fluid in conjunction with the transfer of vacuum pressure to the tissue collection container 32 to maintain a suitable pressure within the patient at the targeted tissue site. In the second configuration, the ultrasound transducer 48 is decoupled from the tissue removal device 4 via withdrawal of the ultrasound transducer from the tissue removal device 4.

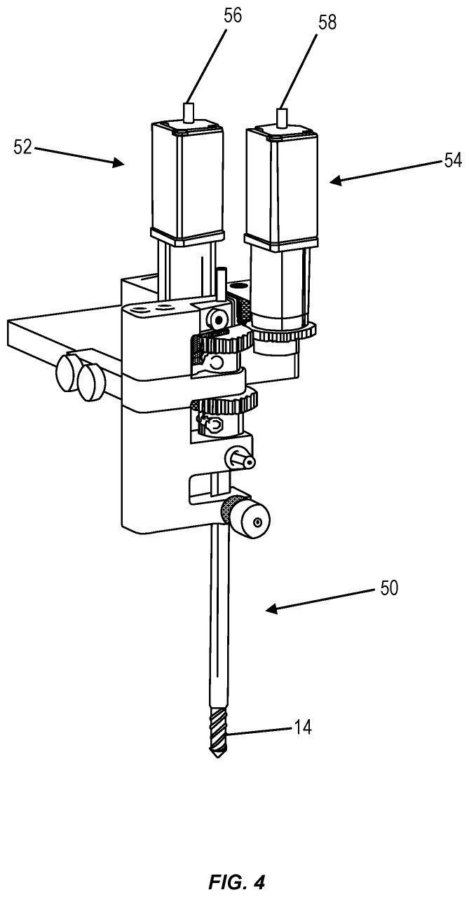

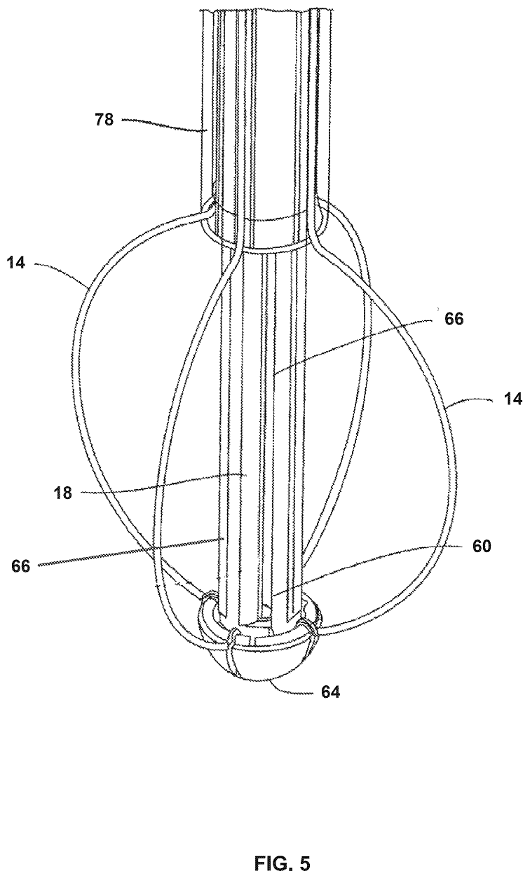

FIG. 4 shows the tissue removal device 4. The tissue removal device 4 includes a probe assembly 50, a tissue cutting wire actuation assembly 52, and a cutting tube actuation assembly 54. As described herein, the tissue cutting wire actuation assembly 52 is drivingly coupled with the tissue cutting wires 14. The tissue cutting wire actuation assembly 52 includes a tissue cutting wire drive input 56 configured to be rotated by the tissue cutting wire drive motor 42 via the tissue cutting wire drive cable assembly 38. As described herein, rotation of the tissue cutting wire drive input 56 in a first direction produces simultaneous rotation and expansion of the tissue cutting wires 14. Rotation of the tissue cutting wire drive input 56 in a second direction (opposite to the first direction) produces simultaneous rotation and contraction of the tissue cutting wires 14. The cutting tube actuation assembly 54 includes a cutting tube actuation input 58 configured to be rotated by the tissue cutting tube drive motor 44 via the tissue cutting tube drive cable assembly 40. As described herein, rotation of the cutting tube actuation input 56 produces rotation of a cutting tube 60 (shown in FIG. 5) of the tissue removal device 4.

FIG. 5 shows a distal end portion of the tissue removal device 4 in an expanded configuration of tissue cutting wires 14. The components of the tissue removal device 4 illustrated in FIG. 5 include the tissue cutting wires 14, the slotted cutting tube 60, a tissue cutting wire support tube 18, and a tissue cutting wire support tube distal member 64. The tissue cutting wire support tube 18 has one or more slots 66. The tissue cutting wire support tube distal member 64 is fixedly attached to the distal end of the tissue cutting wire support tube 18. Distal ends of the tissue cutting wires 14 are attached to the tissue cutting wire support tube distal member 64. Proximal end portions of the tissue cutting wires 14 extend into an annular space between the probe shaft 78 and the tissue cutting wire support tube 18. As described in more detail herein, rotation of the tissue cutting wire drive input 56 in the first direction produces rotation of the combination of the tissue cutting wire support tube 18 and the tissue cutting wires 14 and expansion of the tissue cutting wires 14 radially outwardly from the tissue cutting wire support tube 18. Rotation of the tissue cutting wire drive input 56 in the second direction produces rotation of the combination of the tissue cutting wire support tube 18 and the tissue cutting wires 14 and contraction of the tissue cutting wires 14 radially inwardly towards the tissue cutting wire support tube 18. Rotation of the cutting tube actuation input 58 produces rotation of the slotted cutting tube 60. The slotted cutting tube 60 is disposed within a central lumen of the tissue cutting wire support tube 18. The slotted cutting tube 60 includes slots. The rotation of the slotted cutting tube 60 relative to the tissue cutting wire support tube 18 produces relative movement between the slots of the slotted cutting tube 60 and the slots 66 of the tissue cutting wire support tube 18. The slotted cutting tube 60 has a central lumen through which tissue and/or fluid is aspirated. When the slots of the slotted cutting tube 60 are aligned with the slots 66 of the tissue cutting wire support tube 18, tissue and/or fluid can be drawn into the central lumen of the cutting tube 60 via the vacuum generated by the vacuum source 30. Tissue disposed in the slots 66 can be cut (chopped) via the rotation of the cutting tube 60 relative to the tissue cutting wire support tube 18, thereby reducing the size of the cut tissue pieces to facilitate aspiration of the cut tissue pieces through the central lumen of the cutting tube 60 and the connection tube connecting the central lumen of the cutting tube 60 with the tissue connection container.

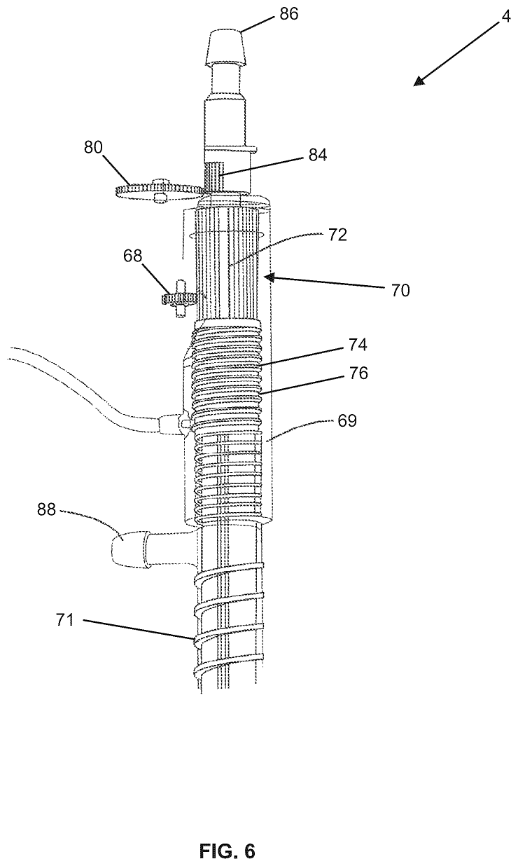

FIG. 6 illustrates components of the tissue removal device 4 related to actuation of the tissue cutting wires 14 and actuation of the slotted cutting tube 60. The tissue removal device 4 includes a base member 69. In the illustrated embodiment, the base member 69 has exterior helical threads 71 for the position of the probe sleeve 78 (shown in FIG. 5) along the base member 69. The tissue cutting wire actuation assembly 52 includes a tissue cutting wire support tube drive gear 68 and an elevator member 70. The elevator member 70 has longitudinally extended outwardly facing gear teeth 72 that are engaged by the tissue cutting wire support tube drive gear 68. Rotation of the tissue cutting wire drive input 56 in the first direction produces corresponding rotations of the tissue cutting wire support tube drive gear 68 and the elevator member 70. The elevator member 70 has a splined inner surface that engages a corresponding splined outer surface of the tissue cutting wire support tube 18 so that the tissue cutting wire support tube 18 and the tissue cutting wires 14 are rotated along with the elevator member 70. The elevator member 70 has exterior surface helical threads 74 that engage mating interior surface helical threads 76 of the base member 69 so that rotation of the elevator member 70 relative to the base member 69 produces longitudinal translation of the elevator member 70 relative to the base member 69. Proximal ends of the tissue cutting wires 14 are attached to the elevator member 70 and the longitudinal translation of the elevator member 70 relative to the base member 69 is used to control expansion and contraction of the tissue cutting wires 14. The cutting tube actuation assembly 54 includes a cutting tube drive gear 80 with exterior gear teeth 82 that engage exterior gear teeth 84 of the slotted cutting tube 60. Rotation of the cutting tube actuation input 58 produces corresponding rotations of the spur gear 80 and the slotted cutting tube 60. The tissue removal device 4 includes an aspiration port 86 in fluid communication with the central lumen of the slotted cutting tube 60 for aspiration cut pieces of tissue and/or fluid from the targeted tissue site. The tissue removal device 4 includes an irrigation port 88 for receiving irrigation fluid for irrigation of the targeted tissue site.

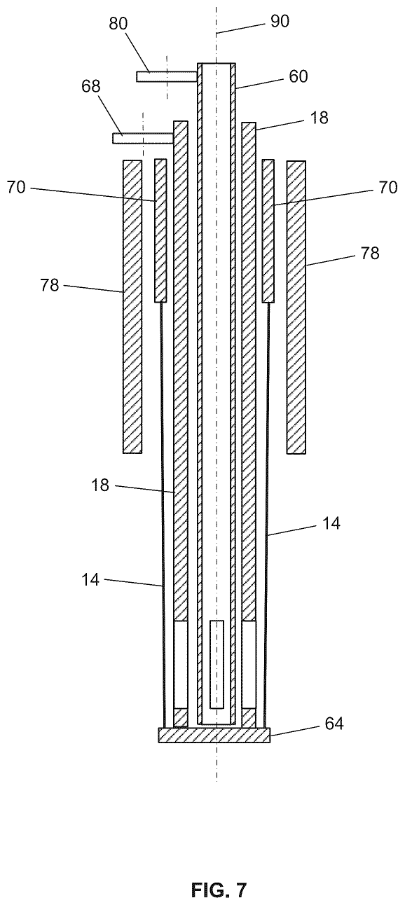

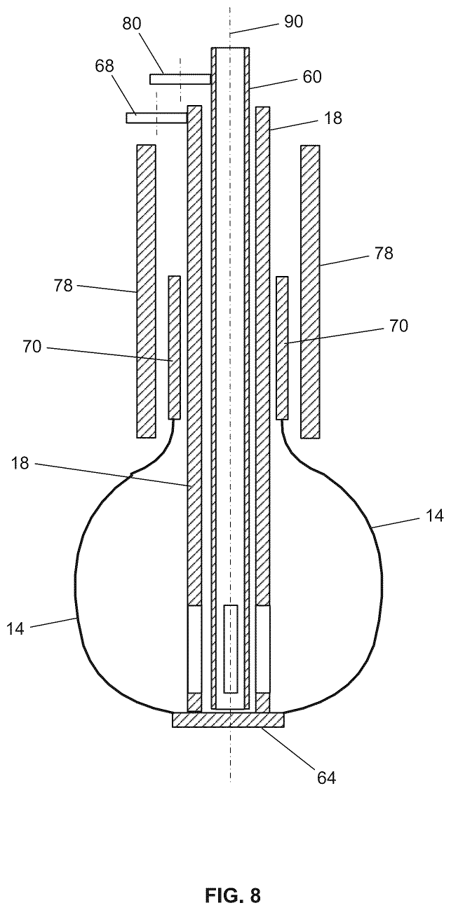

FIG. 7 schematically illustrates tissue cutting wire actuation related components and cutting tube actuation related components of the tissue removal device 4 in a contracted configuration of tissue cutting wires. FIG. 8 schematically illustrates the tissue cutting wire actuation related components and cutting tube actuation related components of the tissue removal device 4 in an expanded configuration of tissue cutting wires. The illustrated components include the slotted cutting tube 60, the cutting tube drive gear 80, the tissue cutting wire support tube 18, the tissue cutting wire support tube drive gear 68, the elevator member 70, the tissue cutting wires 14, the tissue cutting wire support tube distal member 64, and the probe sleeve 78. As described herein, rotation of the cutting tube drive gear 80 produces corresponding rotation of the slotted cutting tube 60. Rotation of the tissue cutting wire support tube drive gear 68 produces a corresponding rotation of the tissue cutting wire support tube 18 around a central probe axis 90. The elevator member 70 and the tissue cutting wire support tube 18 include interfacing spline features by which the elevator member 70 is constrained to rotate with the tissue cutting wire support tube 18 and free to translate longitudinally along the tissue cutting wire support tube 18 parallel to the central probe axis 90. The elevator member 70 and the probe sleeve 78 include interfacing helical threads via which rotation of the elevator member 70 with the tissue cutting wire support tube 18 produces longitudinal translation of the elevator member 70 relative to both the tissue cutting wire support tube 18 and the probe sleeve 78. Rotation of the elevator member 70 in a first direction is used to reconfigure the tissue cutting wires 14 from the contracted configuration illustrated in FIG. 7 to the expanded configuration shown in FIG. 8. Rotation of the elevator member 70 in a second direction (opposite to the first direction) is used to reconfigure the cutting tissue wires 14 from the expanded configuration illustrated in FIG. 8 back to the contracted configuration illustrated in FIG. 7. The expanded diameter of the tissue cutting wires 14 is set by adjusting the distance between the probe sleeve 78 and the base member 69, thereby adjusting the exposed length of the tissue cutting wire support tube 18. The probe sleeve 78 has internal threads engaged with the exterior threads 71 of the base member. The longitudinal position of the probe sleeve 78 relative to the base member 69 is adjusted by rotating the probe sleeve 78 relative to the base member 69.

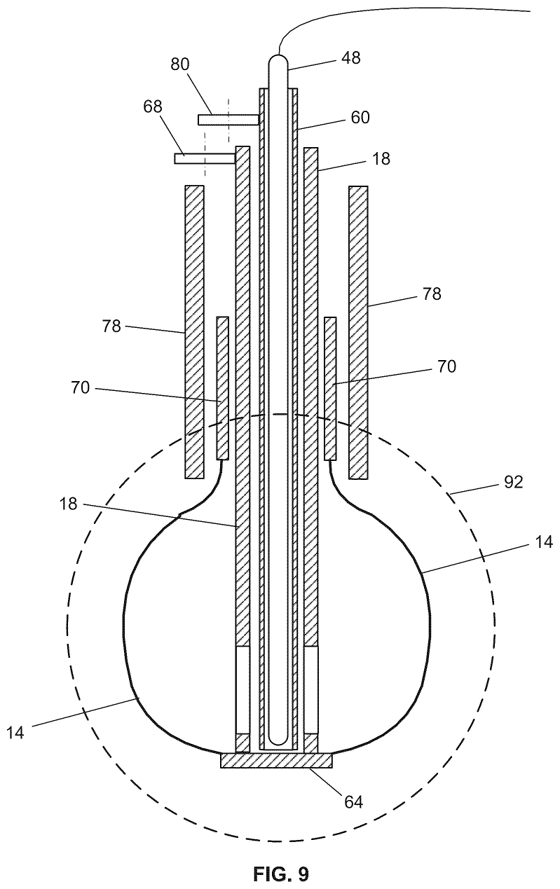

FIG. 9 schematically illustrates the tissue cutting wire actuation related components, cutting tube actuation related components, and the ultrasound transducer 48 of the tissue removal device 4 in an expanded configuration of tissue cutting wires. In the illustrated configuration, the ultrasound transducer 48 is disposed at the distal end of the central lumen of the slotted cutting tube 60 for use in conducting ultrasound imaging of an imaged volume 92 that contains the tissue cutting wires 14 and the targeted tissue during operational phases of the tissue removal device 4. The ultrasound transducer 48 can be removed to enhance tissue and/or fluid aspiration via the central lumen of the slotted cutting tube 60.

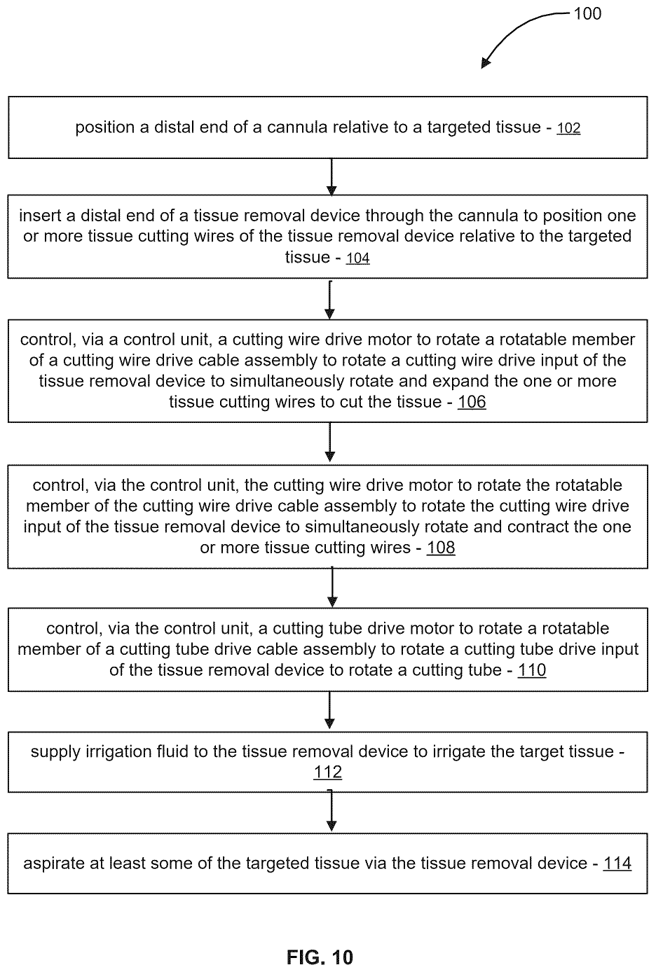

FIG. 10 shows a simplified schematic flow chart of a method 100 of removing a targeted tissue from a patient, in accordance with tissue removal systems and tissue removal methods of the present disclosure. Any suitable tissue removal system, such as the tissue removal system 22 described herein, can be used to practice the method 100.

In act 102, a distal end of a cannula is positioned relative to the targeted tissue. For example, when the targeted tissue is a brain tumor or lesion, a hole can be made through the skull. Any suitable approach can be used to secure the position and orientation of the cannula relative to the skull.

In act 104, a distal end of a tissue removal device is inserted through the cannula to position one or more tissue cutting wires of the tissue removal device adjacent to or within the targeted tissue. For example, the distal end portion of the tissue removal device 4 can be inserted through the cannula to position the tissue cutting wires 14 adjacent to or within the targeted tissue.

In act 106, a tissue cutting wire drive motor is controlled, via a control unit, to rotate a rotatable member of a tissue cutting wire drive cable assembly to rotate a tissue cutting wire drive input of the tissue removal device to simultaneously rotate and expand the one or more tissue cutting wires to cut the targeted tissue. For example, using the tissue removal system 22, the tissue cutting wire drive motor 42 can be controlled by the controller 24 to rotate the rotatable member of the tissue cutting wire drive cable assembly 38 to rotate the tissue cutting wire drive input 56 of the tissue removal device 4 to simultaneously rotate and expand the tissue cutting wires 14 to cut the targeted tissue.

In act 108, the tissue cutting wire drive motor is controlled, via the control unit, to rotate the rotatable member of the tissue cutting wire drive cable assembly to rotate the tissue cutting wire drive input of the tissue removal device to simultaneously rotate and contract the one or more tissue cutting wires. For example, using the tissue removal system 22, the tissue cutting wire drive motor 42 can be controlled by the controller 24 to rotate the rotatable member of the tissue cutting wire drive cable assembly 38 to rotate the tissue cutting wire drive input 56 of the tissue removal device 4 to simultaneously rotate and contract the tissue cutting wires 14.

In act 110, a tissue cutting tube drive motor is controlled, via the control unit, to rotate a rotatable member of a tissue cutting tube drive cable assembly to rotate the tissue cutting tube drive input of the tissue removal device to rotate a tissue cutting tube. For example, using the tissue removal system 22, the tissue cutting tube drive motor 44 can be controlled by the controller 24 to rotate the rotatable member of the tissue cutting tube drive cable assembly 40 to rotate the tissue cutting tube drive input 58 of the tissue removal device 4 to rotate the slotted cutting tube 60.

The method 100 includes irrigation and aspiration of the targeted tissue. In act 110, irrigation fluid is output from the tissue removal device to irrigate the targeted tissue. In act 112, at least some of the irrigation fluid and/or at least some of the targeted tissue is aspirated via the tissue removal device.

In the method 100, the tissue removal device can chop aspirated tissue. For example, the tissue removal device can include a tissue cutting wire support tube and a tissue cutting tube disposed within the tissue cutting wire support tube. The tissue cutting wire support tube can have tissue cutting wire support tube tissue removal openings. The tissue cutting tube can have a tissue cutting tube lumen and tissue cutting tube radial openings configured to cooperate with the tissue cutting wire support tube tissue removal openings to chop tissue and allow aspiration of the chopped tissue through the tissue cutting tube lumen.

The method 100 can include magnetic resonance imaging. For example, the method 100 can further include conducting magnetic resonance imaging of the targeted tissue during operation of the tissue removal device. Each of the tissue removal device, the tissue cutting wire drive cable assembly, and the tissue cutting tube drive cable assembly can be made of one or more magnetic resonance imaging compatible materials.

The method 100 can include ultrasound imaging of the targeted tissue. For example, the method 100 can further include conducting ultrasound imaging of the one or more tissue cutting wires via an ultrasound transducer disposed within the tissue removal device during cutting of the targeted tissue via rotation and expansion of the one or more tissue cutting wires. The method 100 can further include inserting the ultrasound transducer into the tissue removal device prior to the cutting of the targeted tissue via rotation and expansion of the one or more tissue cutting wires. The method 100 can further include removing the ultrasound transducer from the tissue removal device prior to rotation and contraction of the one or more tissue cutting wires and coupling an aspiration assembly to the tissue removal device for aspiration of the targeted tissue.

The method 100 can include applying radio frequency (RF) energy to the targeted tissue. For example, the method 100 can further include operating an RF energy source to supply RF energy to at least one of the tissue cutting wires to apply RF energy to the targeted tissue.

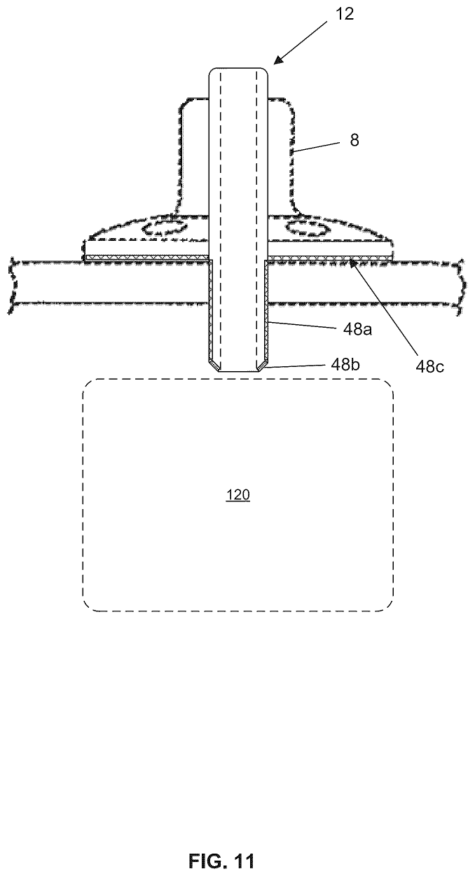

The ultrasound transducer 48 (shown in FIG. 3) can optionally be replaced or supplemented by one or more ultrasound transducers that are incorporated into the cannula 12 and/or the mounting fixture 8 (shown in FIG. 1). For example, FIG. 11 shows ultrasound transducers 48a, 48b integrated with the cannula 12 and one or more ultrasound transducers 48c integrated with the mounting fixture 8. The tissue removal system 22 can include any suitable combination of one or more of the ultrasound transducers 48, 48a, 48b, 48c to image a tissue region 120 during the removal of the tissue via the tissue removal system 22. In the illustrated example, the ultrasound transducer 48a extends around an outer circumferential surface of the cannula 12 and can be configured to direct ultrasound energy and receive reflected ultrasound energy in an oblique direction to image the tissue region 120, which is disposed below the distal end of the cannula 12. The ultrasound transducer 48b extends around a circumferential perimeter of the distal end of the cannular 12 and is oriented to direct ultrasound energy and receive reflected ultrasound energy in an oblique direction to image the tissue region 120. The ultrasound transducer 48c covers a bottom annular surface of the mounting fixture 8 and can be oriented to direct ultrasound energy and receive reflected ultrasound energy to and from the tissue region 120 to image the tissue region 120. The tissue removal system 22 can also be configured to include and operate any suitable combination of the ultrasound transducers 48, 48a, 48b, 48c to disrupt the blood brain barrier to enhance delivery of therapeutics into the brain via infusion directly through the tissue removal device 4 and/or other routes (e.g., intravenous, intranasal).

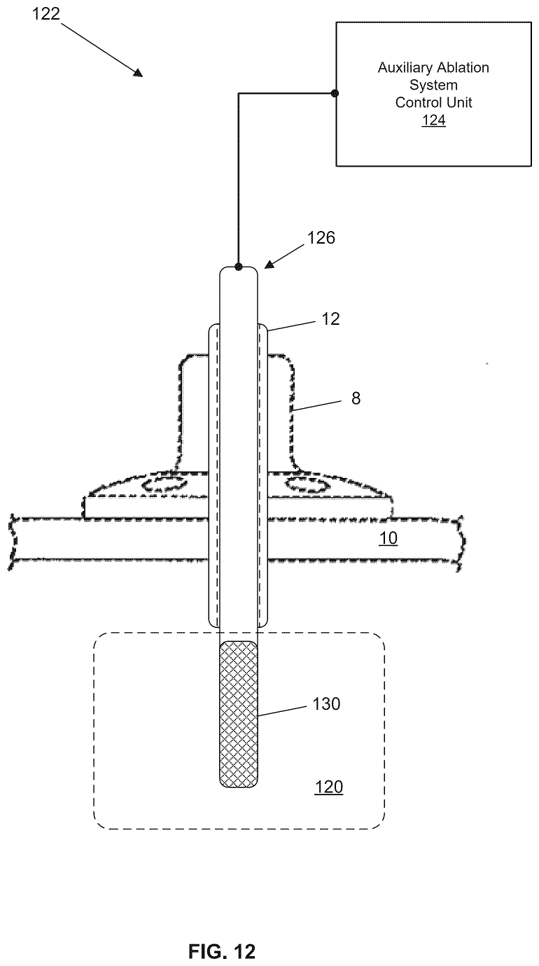

The tissue removal system 22 can be used in conjunction with an auxiliary ablation system to remove the tissue. For example, FIG. 12 schematically illustrates an auxiliary ablation system 122 that can be employed in conjunction with the tissue removal system 22. In the illustrated example, the auxiliary ablation system 122 includes an auxiliary ablation control unit 124 and insertable ablation assembly 126 connected to the control unit 124 via a control cable 128. The insertable ablation assembly 126 includes an auxiliary ablation assembly 130 that is operable by the control unit 124 to ablate the tissue. The insertable assembly 126 is insertable into and partially through the cannula 12 to position the auxiliary ablation assembly 130 adjacent to the tissue to be ablated. The auxiliary ablation assembly 130 can be operable by the control unit 124 to ablate the tissue using any suitable modality. For example, the auxiliary ablation system 122 can be configured to ablate the tissue using Laser Interstitial Thermal Therapy (LITT), Magnetic Resonance Imaging Guided Focused Ultrasound (MRgFUS) ablation, Radiofrequency Ablation (RFA), Microwave Ablation (MWA), Cryoablation Ablation, or Irreversible Electroporation (IRE) ablation. The tissue removal system 22 and the auxiliary ablation system 122 can be used in any suitable sequence.

The following relate to numbered aspects of the invention:
1. A tissue removal system comprising:
   a tissue removal device comprising a tissue cutting wire support tube, a tissue cutting tube, one or more tissue cutting wires, a tissue cutting wire actuation mechanism, and a tissue cutting tube actuation mechanism, wherein the tissue cutting wire support tube has a tissue cutting wire support tube lumen, wherein the tissue cutting tube has a tissue cutting tube lumen, wherein the tissue cutting tube is disposed within the tissue cutting wire support tube lumen, wherein the one or more tissue cutting wires are configured to be rotated to cut a tissue, wherein the tissue cutting wire actuation mechanism comprises a tissue cutting wire drive input, wherein the tissue cutting wire actuation mechanism is configured to rotate and outwardly expand the one or more tissue cutting wires in response to rotation of the tissue cutting wire drive input in a first direction, wherein the tissue cutting wire actuation mechanism is configured to rotate and inwardly contract the one or more tissue cutting wires in response to rotation of the tissue cutting wire drive input in a second direction opposite to the first direction, wherein the tissue cutting tube actuation mechanism comprises a tissue cutting tube drive input, wherein the tissue cutting tube actuation mechanism is configured to rotate the tissue cutting tube in response to rotation of the tissue cutting tube drive input, wherein the tissue cutting wire support tube has tissue cutting wire support tube tissue removal openings, and wherein the tissue cutting tube has tissue cutting tube radial openings configured to cooperate with the tissue cutting wire support tube tissue removal openings to chop tissue and allow aspiration of the chopped tissue through the tissue cutting tube lumen;
   a tissue cutting wire drive cable assembly drivingly coupled with the tissue cutting wire drive input;
   a tissue cutting tube drive cable assembly drivingly coupled with the tissue cutting tube drive input; and
   a control unit comprising a tissue cutting wire drive output, a tissue cutting wire drive motor drivingly coupled with the tissue cutting wire drive output, a tissue cutting tube drive output, and a tissue cutting tube drive motor drivingly coupled with the tissue cutting tube drive output, wherein the tissue cutting wire drive output is configured to be drivingly coupled with the tissue cutting wire drive cable assembly, wherein the tissue cutting tube drive output is configured to be drivingly coupled with the tissue cutting tube drive cable assembly, wherein the control unit is configured to control operation of the tissue cutting wire drive motor to control rotation, expansion, and contraction of the one or more tissue cutting wires, and wherein the control unit is configured to control operation of the tissue cutting tube drive motor to control rotation of the tissue cutting tube.
2. The tissue removal system of aspect 1, wherein each of the tissue removal device, the tissue cutting wire drive cable assembly, and the tissue cutting tube drive cable assembly is made of one or more magnetic resonance imaging compatible materials to accommodate usage of magnetic resonance imaging with the tissue removal device.
3. The tissue removal system of aspect 1, further comprising an ultrasound transducer for imaging the one or more tissue cutting wires during cutting of the tissue via rotation and expansion of the one or more tissue cutting wires.
4. The tissue removal system of aspect 3, wherein the tissue removal device comprises an aspiration port, and wherein the tissue removal system further comprising a tissue aspiration assembly in fluid communication with the aspiration port and operable to aspirate the tissue cut by the one or more tissue cutting wires.
5. The tissue removal system of aspect 4, wherein the ultrasound transducer is insertable into the tissue cutting tube lumen to a position for the imaging of the one or more tissue cutting wires.
6. The tissue removal system of aspect 5, wherein the tissue aspiration assembly comprises a tissue aspiration tube configured to be fluidly coupled with the aspiration port for aspiration of the tissue cut by the one or more tissue cutting wires.
7. The tissue removal system of aspect 6, further comprising an irrigation assembly configured to supply an irrigation fluid, wherein the tissue removal device comprises an irrigation port configured to receive the irrigation fluid, and wherein the tissue removal device is configured to output the irrigation fluid to irrigate the tissue cut by the one or more tissue cutting wires and/or a tissue bordering the tissue cut by the one or more tissue cutting wires.
8. The tissue removal system of aspect 7, wherein each of the tissue removal device, the tissue cutting wire drive cable assembly, the tissue cutting tube drive cable assembly, and the ultrasound transducer is made of one or more magnetic resonance imaging compatible materials to accommodate usage of magnetic resonance imaging with the tissue removal device.
9. The tissue removal system of aspect 7, wherein the irrigation assembly comprises an irrigation fluid source, wherein the control unit is configured to control the irrigation fluid source and an aspiration assembly to synchronize supply of the irrigation fluid to the irrigation port with the aspiration of the tissue cut by the one or more tissue cutting wires.
10. The tissue removal system of any one of aspect 1 through aspect 3, further comprising:
   a tissue aspiration assembly operable to aspirate the tissue cut by the one or more tissue cutting wires; and
   an irrigation assembly configured to supply an irrigation fluid, wherein the tissue removal device comprises an irrigation port configured to receive the irrigation fluid, and wherein the tissue removal device is configured to output the irrigation fluid to irrigate the tissue cut by the one or more tissue cutting wires and/or a tissue bordering the tissue cut by the one or more tissue cutting wires.
11. The tissue removal system of any one of aspect 1 through aspect 9, wherein the tissue cutting wire actuation mechanism is configured to:
   expand the one or more tissue cutting wires by moving proximal ends of the one or more tissue cutting wires toward a distal end of the tissue cutting wire support tube; and
   contract the one or more tissue cutting wires by moving the proximal ends of the one or more tissue cutting wires away from the distal end of the tissue cutting wire support tube.
12. The tissue removal system of any one of aspect 1 through aspect 9, further comprising an energy generator connection operatively coupled with at least one of the one or more tissue cutting wires for transferring radiofrequency (RF) energy from an RF energy source to the at least one of the one or more tissue cutting wires.
13. The tissue removal system of any one of aspect 1 through aspect 9, further comprising a cannula having a distal end and configured to be held in a fixed position relative to the tissue, wherein a distal end of the tissue removal device is insertable through the cannula to position the tissue removal device for removal of the tissue, and wherein the cannula comprises one or more ultrasound transducers of an ultrasound imaging system operable to image the tissue during removal of the tissue via operation of the tissue removal device.
14. The tissue removal system of any one of aspect 1 through aspect 9, further comprising:
   a cannula having a distal end and configured to be held in a fixed position relative to the tissue, wherein a distal end of the tissue removal device is insertable through the cannula to position the tissue removal device for removal of the tissue, and wherein the cannula comprises one or more cannular ultrasound transducers of an ultrasound imaging system operable to image the tissue during removal of the tissue via operation of the tissue removal device; and
   a mounting fixture configured for supporting the tissue removal device relative to the tissue, wherein the mounting fixture comprises one or more mounting fixture ultrasound transducers of the ultrasound imaging system.
15. The tissue removal system of any one of aspect 1 through aspect 9, further comprising:
   a cannula having a distal end and configured to be held in a fixed position relative to the tissue, wherein a distal end of the tissue removal device is insertable through the cannula to position the tissue removal device for removal of the tissue; and
   an auxiliary ablation device comprising an auxiliary ablation assembly operable to ablate the tissue, wherein the auxiliary ablation assembly is insertable through the cannula to position the auxiliary ablation assembly adjacent the tissue for ablation of the tissue by the auxiliary ablation assembly.
16. The tissue removal system of aspect 15, wherein the auxiliary ablation assembly comprises a Laser Interstitial Thermal Therapy (LITT) ablation assembly operable to ablate the tissue via LITT.
17. The tissue removal system of aspect 15, wherein the auxiliary ablation assembly comprises a Magnetic Resonance Imaging Guided Focused Ultrasound (MRgFUS) ablation assembly operable to ablate the tissue via MRgFUS.
18. The tissue removal system of aspect 15, wherein the auxiliary ablation assembly comprises a Radiofrequency Ablation (RFA) assembly operable to ablate the tissue via RFA.
19. The tissue removal system of aspect 15, wherein the auxiliary ablation assembly comprises a Microwave Ablation (MWA) assembly operable to ablate the tissue via MWA.
20. The tissue removal system of aspect 15, wherein the auxiliary ablation assembly comprises a Cryoablation Ablation assembly operable to ablate the tissue via cyroablation.
21. The tissue removal system of aspect 15, wherein the auxiliary ablation assembly comprises an Irreversible Electroporation (IRE) assembly operable to ablate the tissue via IRE.
22. A method of removing a targeted tissue from a patient, the method comprising:
   positioning a distal end of a cannula relative to the targeted tissue;
   inserting a distal end of a tissue removal device through the cannula to position one or more tissue cutting wires of the tissue removal device adjacent to or within the targeted tissue;
   controlling, via a control unit, a tissue cutting wire drive motor to rotate a rotatable member of a tissue cutting wire drive cable assembly to rotate a tissue cutting wire drive input of the tissue removal device to simultaneously rotate and expand the one or more tissue cutting wires to cut the targeted tissue;
   controlling, via the control unit, the tissue cutting wire drive motor to rotate the rotatable member of the tissue cutting wire drive cable assembly to rotate the tissue cutting wire drive input of the tissue removal device to simultaneously rotate and contract the one or more tissue cutting wires;
   outputting irrigation fluid from the tissue removal device to irrigate the targeted tissue; and
   aspirating at least some of the targeted tissue via the tissue removal device.
23. The method of aspect 22, wherein:
   the tissue removal device comprises a tissue cutting wire support tube and a tissue cutting tube disposed within the tissue cutting wire support tube;
   the tissue cutting wire support tube has tissue cutting wire support tube tissue removal openings; and
   the tissue cutting tube has a tissue cutting tube lumen and tissue cutting tube radial openings configured to cooperate with the tissue cutting wire support tube tissue removal openings to chop tissue and allow aspiration of the chopped tissue through the tissue cutting tube lumen.
   24. The method of aspect 23, further comprising controlling, via the control unit, a tissue cutting tube drive motor to rotate a rotatable member of a tissue cutting tube drive cable assembly to rotate a tissue cutting tube drive input of the tissue removal device to rotate the tissue cutting tube.
25. The method of any one of aspect 22 through aspect 24, further comprising conducting magnetic resonance imaging of the targeted tissue during operation of the tissue removal device, wherein each of the tissue removal device, the tissue cutting wire drive cable assembly, and the tissue cutting tube drive cable assembly is made of one or more magnetic resonance imaging compatible materials.
26. The method of any one of aspect 22 through aspect 24, further comprising conducting ultrasound imaging of the one or more tissue cutting wires via an ultrasound transducer disposed within the tissue removal device during cutting of the targeted tissue via rotation and expansion of the one or more tissue cutting wires.
27. The method of aspect 26, further comprising inserting the ultrasound transducer into the tissue removal device prior to the cutting of the targeted tissue via rotation and expansion of the one or more tissue cutting wires.
28. The method of aspect 27, further comprising:
   removing the ultrasound transducer from the tissue removal device prior to rotation and contraction of the one or more tissue cutting wires; and
   coupling an aspiration assembly to the tissue removal device for aspiration of the targeted tissue cut by the one or more tissue cutting wires.
29. The method of any one of aspect 22 through aspect 24, further comprising operating a radio frequency (RF) energy source to supply RF energy to at least one of the tissue cutting wires to apply RF energy to the targeted tissue.
30. The method of any one of aspect 22 through aspect 24, further comprising imaging the tissue during removal of the tissue via operation of the tissue removal device via an ultrasonic imaging system comprising one or more ultrasound transducers mounted to the cannula.
31. The method of any one of aspect 22 through aspect 24, further comprising imaging the tissue during removal of the tissue via operation of the tissue removal device via an ultrasonic imaging system comprising one or more cannula ultrasound transducers mounted to the cannula and one or more mounting fixture ultrasound transducers mounted to a mounting fixture configured to support the tissue removal device relative to the tissue.
32. The method of any one of aspect 22 through aspect 24, further comprising operating an auxiliary ablation device to ablate the tissue, wherein the auxiliary ablation device comprises an auxiliary ablation assembly that is positioned relative to the tissue through the cannula.
33. The method of aspect 32, wherein the auxiliary ablation assembly comprises a Laser Interstitial Thermal Therapy (LITT) ablation assembly operable to ablate the tissue via LITT.
34. The method of aspect 32, wherein the auxiliary ablation assembly comprises a Magnetic Resonance Imaging Guided Focused Ultrasound (MRgFUS) ablation assembly operable to ablate the tissue via MRgFUS.
35. The method of aspect 32, wherein the auxiliary ablation assembly comprises a Radiofrequency Ablation (RFA) assembly operable to ablate the tissue via RFA.
36. The method of aspect 32, wherein the auxiliary ablation assembly comprises a Microwave Ablation (MWA) assembly operable to ablate the tissue via MWA.
37. The method of aspect 32, wherein the auxiliary ablation assembly comprises a Cryoablation Ablation assembly operable to ablate the tissue via cyroablation.
38. The method of aspect 32, wherein the auxiliary ablation assembly comprises an Irreversible Electroporation (IRE) assembly operable to ablate the tissue via IRE.

Other variations are within the spirit of the tissue removal systems and tissue removal methods of the present disclosure. Thus, while the tissue removal systems and tissue removal methods of the present disclosure may be compatible with various modifications and alternative constructions, certain illustrated tissue removal systems and tissue removal methods of the present disclosure thereof are shown in the drawings and have been described above in detail. It should be understood, however, that there is no intention to limit the tissue removal systems and tissue removal methods of the present disclosure to the specific form or forms disclosed, but on the contrary, the intention is to cover all modifications, alternative constructions, and equivalents falling within the spirit and scope of the tissue removal systems and tissue removal methods of the present disclosure, as defined in the appended claims.

The use of the terms “a” and “an” and “the” and similar referents in the context of describing the tissue removal systems and tissue removal methods of the present disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate tissue removal systems and tissue removal methods of the present disclosure and does not pose a limitation on the scope of the tissue removal systems and tissue removal methods of the present disclosure unless otherwise claimed. No language in the specification should be construed as indicating any non-claimed element as essential to the practice of the tissue removal systems and tissue removal methods of the present disclosure.

Preferred tissue removal systems and tissue removal methods of the present disclosure are described herein, including the best mode known to the inventors for carrying out the tissue removal systems and tissue removal methods of the present disclosure. Variations of those preferred tissue removal systems and tissue removal methods of the present disclosure may become apparent to those of ordinary skill in the art upon reading the foregoing description. The inventors expect skilled artisans to employ such variations as appropriate, and the inventors intend for the tissue removal systems and tissue removal methods of the present disclosure to be practiced otherwise than as specifically described herein. Accordingly, the tissue removal systems and tissue removal methods of the present disclosure include all modifications and equivalents of the subject matter recited in the claims appended hereto as permitted by applicable law. Moreover, any combination of the above-described elements in all possible variations thereof is encompassed by the tissue removal systems and tissue removal methods of the present disclosure unless otherwise indicated herein or otherwise clearly contradicted by context.

1. A tissue removal system comprising:
a tissue removal device comprising a tissue cutting wire support tube, a tissue cutting tube, one or more tissue cutting wires, a tissue cutting wire actuation mechanism, and a tissue cutting tube actuation mechanism, wherein the tissue cutting wire support tube has a tissue cutting wire support tube lumen, wherein the tissue cutting tube has a tissue cutting tube lumen, wherein the tissue cutting tube is disposed within the tissue cutting wire support tube lumen, wherein the one or more tissue cutting wires are configured to be rotated to cut a tissue, wherein the tissue cutting wire actuation mechanism comprises a tissue cutting wire drive input, wherein the tissue cutting wire actuation mechanism is configured to rotate and outwardly expand the one or more tissue cutting wires in response to rotation of the tissue cutting wire drive input in a first direction, wherein the tissue cutting wire actuation mechanism is configured to rotate and inwardly contract the one or more tissue cutting wires in response to rotation of the tissue cutting wire drive input in a second direction opposite to the first direction, wherein the tissue cutting tube actuation mechanism comprises a tissue cutting tube drive input, wherein the tissue cutting tube actuation mechanism is configured to rotate the tissue cutting tube in response to rotation of the tissue cutting tube drive input, wherein the tissue cutting wire support tube has tissue cutting wire support tube tissue removal openings, and wherein the tissue cutting tube has tissue cutting tube radial openings configured to cooperate with the tissue cutting wire support tube tissue removal openings to chop tissue and allow aspiration of the chopped tissue through the tissue cutting tube lumen;
a tissue cutting wire drive cable assembly drivingly coupled with the tissue cutting wire drive input;
a tissue cutting tube drive cable assembly drivingly coupled with the tissue cutting tube drive input; and
a control unit comprising a tissue cutting wire drive output, a tissue cutting wire drive motor drivingly coupled with the tissue cutting wire drive output, a tissue cutting tube drive output, and a tissue cutting tube drive motor drivingly coupled with the tissue cutting tube drive output, wherein the tissue cutting wire drive output is configured to be drivingly coupled with the tissue cutting wire drive cable assembly, wherein the tissue cutting tube drive output is configured to be drivingly coupled with the tissue cutting tube drive cable assembly, wherein the control unit is configured to control operation of the tissue cutting wire drive motor to control rotation, expansion, and contraction of the one or more tissue cutting wires, and wherein the control unit is configured to control operation of the tissue cutting tube drive motor to control rotation of the tissue cutting tube.

2. The tissue removal system of claim 1, wherein each of the tissue removal device, the tissue cutting wire drive cable assembly, and the tissue cutting tube drive cable assembly is made of one or more magnetic resonance imaging compatible materials to accommodate usage of magnetic resonance imaging with the tissue removal device.

3. The tissue removal system of claim 1, further comprising an ultrasound transducer for imaging the one or more tissue cutting wires during cutting of the tissue via rotation and expansion of the one or more tissue cutting wires.

4. The tissue removal system of claim 3, wherein the tissue removal device comprises an aspiration port, and wherein the tissue removal system further comprising a tissue aspiration assembly in fluid communication with the aspiration port and operable to aspirate the tissue cut by the one or more tissue cutting wires.

5. The tissue removal system of claim 4, wherein the ultrasound transducer is insertable into the tissue cutting tube lumen to a position for the imaging of the one or more tissue cutting wires.

6. The tissue removal system of claim 5, wherein the tissue aspiration assembly comprises a tissue aspiration tube configured to be fluidly coupled with the aspiration port for aspiration of the tissue cut by the one or more tissue cutting wires.

7. The tissue removal system of claim 6, further comprising an irrigation assembly configured to supply an irrigation fluid, wherein the tissue removal device comprises an irrigation port configured to receive the irrigation fluid, and wherein the tissue removal device is configured to output the irrigation fluid to irrigate the tissue cut by the one or more tissue cutting wires and/or a tissue bordering the tissue cut by the one or more tissue cutting wires.

8. The tissue removal system of claim 7, wherein each of the tissue removal device, the tissue cutting wire drive cable assembly, the tissue cutting tube drive cable assembly, and the ultrasound transducer is made of one or more magnetic resonance imaging compatible materials to accommodate usage of magnetic resonance imaging with the tissue removal device.

9. The tissue removal system of claim 7, wherein the irrigation assembly comprises an irrigation fluid source, wherein the control unit is configured to control the irrigation fluid source and an aspiration assembly to synchronize supply of the irrigation fluid to the irrigation port with the aspiration of the tissue cut by the one or more tissue cutting wires.

10. The tissue removal system of claim 1, further comprising:
a tissue aspiration assembly operable to aspirate the tissue cut by the one or more tissue cutting wires; and
an irrigation assembly configured to supply an irrigation fluid, wherein the tissue removal device comprises an irrigation port configured to receive the irrigation fluid, and wherein the tissue removal device is configured to output the irrigation fluid to irrigate the tissue cut by the one or more tissue cutting wires and/or a tissue bordering the tissue cut by the one or more tissue cutting wires.

11. The tissue removal system of claim 1, wherein the tissue cutting wire actuation mechanism is configured to:
expand the one or more tissue cutting wires by moving proximal ends of the one or more tissue cutting wires toward a distal end of the tissue cutting wire support tube; and
contract the one or more tissue cutting wires by moving the proximal ends of the one or more tissue cutting wires away from the distal end of the tissue cutting wire support tube.

12. The tissue removal system of claim 1, further comprising an energy generator connection operatively coupled with at least one of the one or more tissue cutting wires for transferring radiofrequency (RF) energy from an RF energy source to the at least one of the one or more tissue cutting wires.

13. The tissue removal system of claim 1, further comprising a cannula having a distal end and configured to be held in a fixed position relative to the tissue, wherein a distal end of the tissue removal device is insertable through the cannula to position the tissue removal device for removal of the tissue, and wherein the cannula comprises one or more ultrasound transducers of an ultrasound imaging system operable to image the tissue during removal of the tissue via operation of the tissue removal device.

14. The tissue removal system of claim 1, further comprising:
a cannula having a distal end and configured to be held in a fixed position relative to the tissue, wherein a distal end of the tissue removal device is insertable through the cannula to position the tissue removal device for removal of the tissue, and wherein the cannula comprises one or more cannular ultrasound transducers of an ultrasound imaging system operable to image the tissue during removal of the tissue via operation of the tissue removal device; and
a mounting fixture configured for supporting the tissue removal device relative to the tissue, wherein the mounting fixture comprises one or more mounting fixture ultrasound transducers of the ultrasound imaging system.

15. The tissue removal system of claim 1, further comprising:
a cannula having a distal end and configured to be held in a fixed position relative to the tissue, wherein a distal end of the tissue removal device is insertable through the cannula to position the tissue removal device for removal of the tissue; and
an auxiliary ablation device comprising an auxiliary ablation assembly operable to ablate the tissue, wherein the auxiliary ablation assembly is insertable through the cannula to position the auxiliary ablation assembly adjacent the tissue for ablation of the tissue by the auxiliary ablation assembly.

16. The tissue removal system of claim 15, wherein the auxiliary ablation assembly comprises a Laser Interstitial Thermal Therapy (LITT) ablation assembly operable to ablate the tissue via LITT.

17. The tissue removal system of claim 15, wherein the auxiliary ablation assembly comprises a Magnetic Resonance Imaging Guided Focused Ultrasound (MRgFUS) ablation assembly operable to ablate the tissue via MRgFUS.

18. The tissue removal system of claim 15, wherein the auxiliary ablation assembly comprises a Radiofrequency Ablation (RFA) assembly operable to ablate the tissue via RFA.

19. The tissue removal system of claim 15, wherein the auxiliary ablation assembly comprises a Microwave Ablation (MWA) assembly operable to ablate the tissue via MWA.

20. The tissue removal system of claim 15, wherein the auxiliary ablation assembly comprises a Cryoablation Ablation assembly operable to ablate the tissue via cyroablation.

21. The tissue removal system of claim 15, wherein the auxiliary ablation assembly comprises an Irreversible Electroporation (IRE) assembly operable to ablate the tissue via IRE.

22-38. (canceled)
